(19) United States
(12) Patent Application Publication
(10) Pub. No.: US 2020/0359929 A1
(43) Pub. Date: Nov. 19, 2020

(54) PULSE WAVE PROPAGATION VELOCITY MEASUREMENT DEVICE AND METHOD FOR SAME

(71) Applicant: Taiyo Yuden Co., Ltd., Chuo-ku, Tokyo (JP)

(72) Inventors: Yuka MAEDA, Tsukuba-shi (JP); Takashi ISHIGURO, Takasaki-shi (JP); Keiichi KOBAYASHI, Takasaki-shi (JP)

(21) Appl. No.: 16/963,092

(22) PCT Filed: Jan. 19, 2019
(86) PCT No.: PCT/JP2019/001568

§ 371 Date: Jul. 17, 2020

(30) Foreign Application Priority Data
Jan. 31, 2018 (JP) 2018-015080

(51) Int. Cl. A61B 5/0478; A61B 5/0484; A61B 5/048; A61B 5/11

(52) U.S. Cl. CPC A61B 5/0478; A61B 5/04845; A61B 5/7264; A61B 5/1102; A61B 5/048

(57) ABSTRACT

Pulse waves and ballistocardiac waves propagate to a piezoelectric vibration sensor 110 attached to a seating surface 102 of a chair 100, and are output after being converted to electrical signals. Thereafter, the electrical signals are filtered by a low-pass digital filter 202P and a band-pass digital filter 202B, with the pulse waves GP output from the low-pass digital filters 202P and the ballistocardiac waves GB output from the band-pass digital filter 202B. These pulse waves GP and ballistocardiac waves GB are processed by absolute value circuits 212P, 212B and low-pass filters 214P, 214B of envelope-processing circuits 210P, 210B, to obtain envelopes. Then, pulse wave propagation velocity PWV is calculated based on the difference between the peaks of the obtained envelopes. The pulse waves/their velocity can be detected/obtained even when the piezoelectric vibration sensor is not attached directly to the human body.

### TECHNICAL FIELD

The present invention relates to a pulse wave propagation velocity measurement device, and a method for the same, to measure the velocity of pulse waves representing blood pressure/volume changes occurring in the vascular system due to the beating of the heart.

### BACKGROUND ART

Prior arts relating to pulse waves include, for example, the “arteriosclerosis evaluation device” described in Patent Literature 1 below. This device accurately separates incident waves and reflected waves so that the different degrees of arteriosclerosis among individuals can be evaluated with precision, where the specifics involve using a piezoelectric transducer to detect the pulse waves traveling through the arteries in the subject's neck as displacement signals, while also measuring the blood flow rates in the arteries using the probe of an ultrasonic diagnostic device. Then, the blood flow rates obtained by the probe of the ultrasonic diagnostic device are converted to displacement signals to obtain incident waves, after which the incident waves are subtracted from the displacement signals detected by the piezoelectric transducer to obtain reflected waves, and additionally the amplitude strengths of the incident waves and reflected waves are used to evaluate the vascular function of the body.

However, it would be more convenient and advantageous if pulse waves can be detected to learn about one's state of health without having to attach a piezoelectric sensor directly to his or her body. For example, it would be extremely useful in today's aging society if one's pulse waves are detected daily while sitting in a chair, car seat, etc., so that the person can learn about his or her state of health of the circulatory system, nervous system, etc. In particular, it is important to detect arteriosclerosis early because it can cause cerebral infarction, aortic dissection, and other serious conditions. Recently in the medical field, pulse wave propagation velocity (PWV) is drawing attention as an indicator of this arteriosclerosis, and is increasingly measured as an option as part of complete medical checkups and health screenings.

### BACKGROUND ART LITERATURE

### Patent Literature

Patent Literature 1: International Patent Laid-open No. 2010/024417, Pamphlet

### SUMMARY OF THE INVENTION

### Problems to be Solved by the Invention

It should be noted that the inventors of the invention under the present application for patent previously proposed a technique for measuring pulse wave propagation velocity, which is a technique whereby piezoelectric vibration sensors are fixed at two locations on the body where arteries are present near the body surface, and then the pulse wave propagation velocity is measured from the time difference between the detections of pulse waves at these two locations. This technique is shown in FIG. 7 (A), where piezoelectric vibration sensors 10, 12 are attached to the fingertip and the wrist. A favorable example of the piezoelectric vibration sensors 10, 12 is the vibration waveform sensor disclosed in International Patent Laid-open No. 2016/167202, Pamphlet, for instance. The pulse wave propagation velocity is obtained by measuring the time difference between the detections of pulse waves by these two piezoelectric vibration sensors 10, 12. FIG. 7 (B) shows the pulse wave signals detected by the two piezoelectric vibration sensors 10, 12, while FIG. 7 (C) shows the pulse wave propagation velocity obtained from FIG. 7 (B).

When these piezoelectric vibration sensors for detecting the pulse wave propagation velocity are attached to, for example, a chair, which is an item of daily life used every day, the vibrations of pulse waves generating in the body transmit through the chair at a sonic speed if the chair is made of a hard material. This presents a problem in that crosstalk will occur between the pulse wave signals detected at different locations. A means for reducing the crosstalk involves a method of inserting slits in the chair; while some effects can be expected, however, doing so is not realistic from the viewpoint of chair design.

The present invention focuses on this point, and an object of the present invention is to detect pulse waves and obtain their velocity in a favorable manner even when the piezoelectric vibration sensor is attached not directly, but indirectly, to the human body. Another object of the present invention is to detect pulse waves and obtain their velocity in a favorable manner even when only one piezoelectric vibration sensor is used.

### Means for Solving the Problems

The pulse wave propagation velocity measurement device proposed by the present invention is a pulse wave propagation velocity measurement device that obtains the pulse wave propagation velocity in people based on output vibration waveforms of a piezoelectric vibration sensor attached to a surface contacted by the human body, wherein such device is characterized in that it comprises: a first filtering means for isolating pulse waves from the output vibration waveforms of the piezoelectric vibration sensor; a second filtering means for isolating ballistocardiac waves from the output vibration waveforms of the piezoelectric vibration sensor; and a calculation means for calculating the pulse wave propagation velocity by utilizing the pulse waves obtained by the first filtering means and the ballistocardiac waves obtained by the second filtering means.

According to a preferred embodiment, the pulse wave propagation velocity measurement device is characterized in that the first filtering means isolates frequency range components of 4 Hz or lower from the output vibration waveforms of the piezoelectric vibration sensor, while the second filtering means isolates frequency range components of 10 Hz or higher but no higher than 33 Hz from the output vibration waveforms of the piezoelectric vibration sensor. According to another embodiment, the pulse wave propagation velocity measurement device is characterized in that the calculation means comprises: a first envelope-processing means for obtaining the envelopes of the pulse waves obtained by the first filtering means; a second envelope-processing means for obtaining the envelopes of the ballistocardiac waves obtained by the second filtering means; and a velocity calculation means for calculating pulse wave propagation velocity by utilizing the peaks of the envelopes obtained by the first and second envelope-processing means.

According to another embodiment, the pulse wave propagation velocity measurement device is characterized in that the velocity calculation means obtains the time difference between the pulse wave and the ballistocardiac wave from the peaks of the envelopes, while also calculating “Pulse wave propagation velocity=Blood vessel length/(Time difference between the pulse wave and the ballistocardiac wave)” with respect to the blood vessel length of the path along which the pulse waves travel. According to yet another embodiment, the pulse wave propagation velocity measurement device is characterized in that the first and second envelope-processing means are each constituted by an absolute value circuit and a low-pass filter, where the cutoff frequency of the low-pass filter is set to 1.5 Hz or higher but no higher than 4 Hz. According to yet another embodiment, the pulse wave propagation velocity measurement device is characterized in that it obtains the pulse waves and ballistocardiac waves from the output vibration waveforms of one piezoelectric vibration sensor. Alternatively, the pulse wave propagation velocity measurement device is characterized in that it obtains the pulse waves and ballistocardiac waves from the output vibration waveforms of different piezoelectric vibration sensors.

The method for pulse wave propagation velocity measurement proposed by the present invention is a method for pulse wave propagation velocity measurement that obtains the pulse wave propagation velocity in people based on the output vibration waveforms of a piezoelectric vibration sensor attached to a surface contacted by the human body, wherein such method is characterized in that it comprises: a first step in which pulse waves are isolated from the output vibration waveforms of the piezoelectric vibration sensor; a second step in which ballistocardiac waves are isolated from the output vibration waveforms of the piezoelectric vibration sensor; and a third step in which the pulse wave propagation velocity is calculated by utilizing the pulse waves obtained in the first step and the ballistocardiac waves obtained in the second step.

According to a preferred embodiment, the method for pulse wave propagation velocity measurement is characterized in that the first step isolates frequency range components of 4 Hz or lower from the output vibration waveforms of the piezoelectric vibration sensor, while the second step isolates frequency range components of 10 Hz or higher but no higher than 33 Hz from the output vibration waveforms of the piezoelectric vibration sensor. According to another embodiment, the method for pulse wave propagation velocity measurement is characterized in that the third step comprises: a first envelope-processing step in which the envelopes of the pulse waves obtained in the first step are obtained; a second envelope-processing step in which the envelopes of the ballistocardiac waves obtained in the second step are obtained; and a velocity calculation step in which the pulse wave propagation velocity is calculated by utilizing the peaks of the envelopes obtained in the first and second envelope-processing steps.

According to another embodiment, the method for pulse wave propagation velocity measurement is characterized in that the velocity calculation step obtains the time difference between the pulse wave and the ballistocardiac wave from the peaks of the envelopes, while also calculating “Pulse wave propagation velocity=Blood vessel length/(Time difference between the pulse wave and the ballistocardiac wave)” with respect to the blood vessel length of the path along which the pulse waves travel. According to yet another embodiment, the method for pulse wave propagation velocity measurement is characterized in that it obtains the pulse waves and ballistocardiac waves from the output vibration waveforms of one piezoelectric vibration sensor. Alternatively, the method for pulse wave propagation velocity measurement is characterized in that it obtains the pulse waves and ballistocardiac waves from the output vibration waveforms of different piezoelectric vibration sensors. The aforementioned and other objects, characteristics and benefits of the present invention are made clear by the detailed explanations provided below and the drawings attached hereto.

### Effects of the Invention

According to the present invention, pulse waves and ballistocardiac waves are isolated, by the filtering means, from the output vibration waveforms of the piezoelectric vibration sensor installed on a surface contacted by the human body, and these waves are utilized to calculate pulse wave propagation velocity; accordingly, pulse wave propagation velocity can be obtained in a favorable manner without having to attach the piezoelectric vibration sensor directly to the human body.

### BRIEF DESCRIPTION OF THE DRAWINGS

FIG. 1 Graphs showing examples of various waveforms due to the beating of the heart.

FIG. 2 A drawing showing the placement of the piezoelectric vibration sensor in Example 1 of the present invention.

FIG. 3 Block diagrams showing the configurations of measurement circuits in the aforementioned example.

FIG. 4 Layout drawings of the piezoelectric vibration sensor on a chair, and graphs showing measurement examples of pulse waves and ballistocardiac waves, in the aforementioned example.

FIG. 5 Graphs showing the relationships between the peaks of pulse waves and ballistocardiac waves before and after envelope processing in the aforementioned example.

FIG. 6 Graph of the pulse waves and ballistocardiac waves in FIG. 5 above, and a graph showing the relationship between the pulse wave propagation velocity and its filter cutoff frequency obtained from the graphs.

FIG. 7 A layout drawing of the conventional use of two piezoelectric vibration sensors, and a graph showing a measurement example of pulse waves.

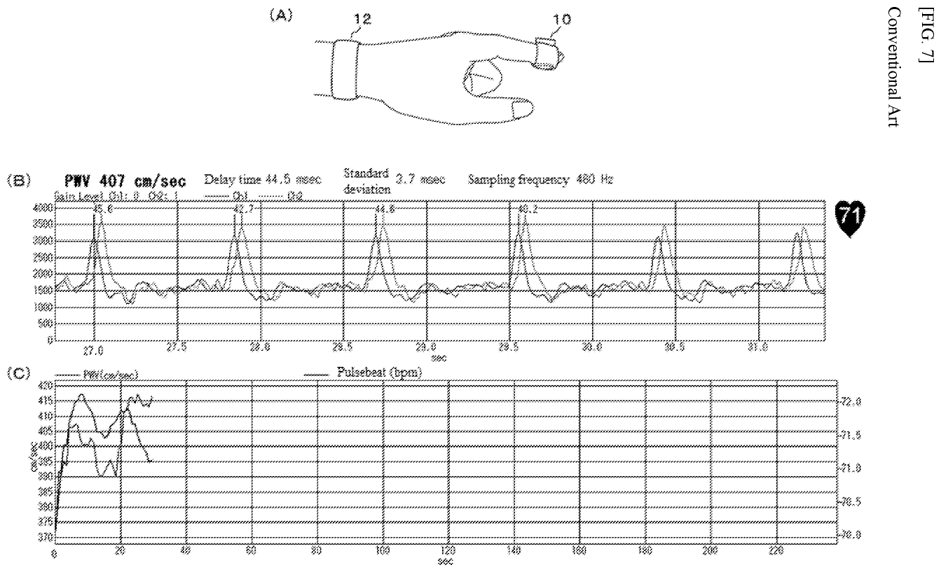

### MODE FOR CARRYING OUT THE INVENTION

The best modes for carrying out the present invention are explained in detail below based on examples.

### Example 1

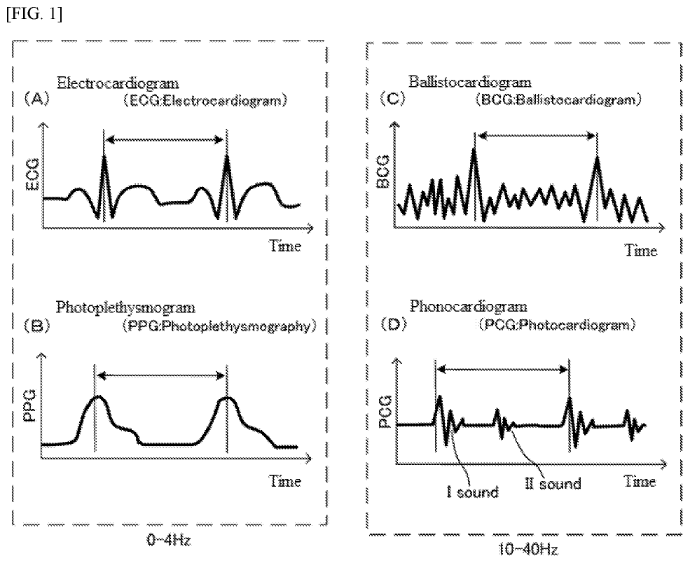

First, the basic concept of the present invention is explained by referring to FIG. 1. FIG. 1 shows major signals relating to the heartbeat and pulsebeat. First, FIG. 1 (A) is an electrocardiogram that records the electrical activities of the heart as waveforms through the body surface. The largest R wave is a waveform that occurs when the ventricles are excited by an impulse traveling from the atria via the impulse conduction system, and the heart rate can be calculated from the interval between the peaks of R waves. FIG. 1 (B) is a photoplethysmogram that measures pulse waves representing inner pressure changes or volume changes (outer diameter changes) in blood vessels that occur according to the beating of the heart, by using the attenuation of transmitted light or reflected light based on hemoglobin concentration.

FIG. 1 (C) is a ballistocardiogram that shows the micro-vibrations of the human body occurring in accordance with the physical movements of the heart, or specifically vibrations from the actions of the heart valves that are transmitted through the skeletal bones, etc., where this transmission is faster than that of the aforementioned pulse waves. Peaks of ballistocardiac waves occur with slight delays from the peaks of electrocardiac waves shown in FIG. 1 (A) or those of pulse waves shown in FIG. 1 (B). FIG. 1 (D) is a phonocardiogram that shows electrical signals that have been converted from the sounds generated by the vibrations of the valve leaflets due to the beating of the heart. Major peaks include the first sound (Isound) that generates when the heart contracts, and the second sound (IIsound) that generates when the heart expands.

Among these, electrocardiac waveforms and photoplethysmic waves have a frequency range of approx. 0 to 4 Hz, while ballistocardiac waveforms and phonocardiac waveforms have a frequency range of 10 to 40 Hz. Accordingly, electrocardiac waveforms or photoplethysmic waves can be differentiated from ballistocardiac waveforms or phonocardiac waveforms by means of range separation. Under the present invention, range separation is performed on the signal waveforms obtained using a piezoelectric vibration sensor, to separate pulse waveforms and ballistocardiac waveforms.

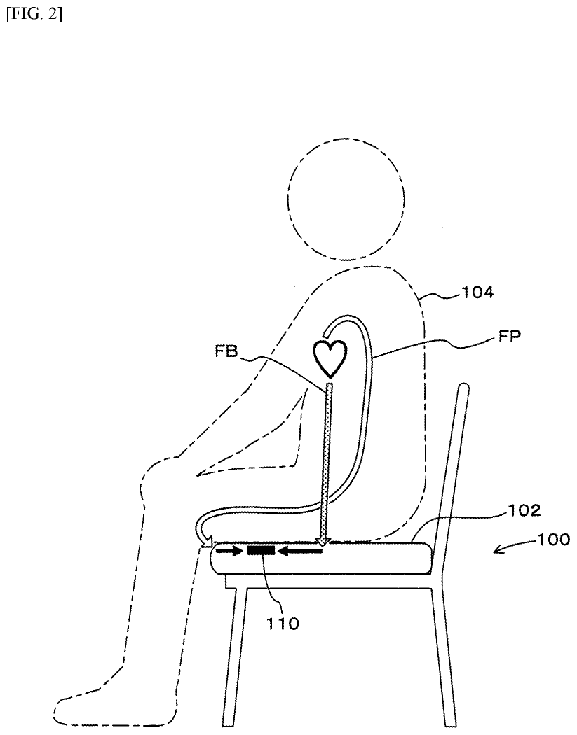

Next, Example 1 of the present invention is explained by also referring to FIG. 2 to FIG. 5. FIG. 2 shows the specific installation of a piezoelectric vibration sensor in this example, where the piezoelectric vibration sensor 110 is installed at an appropriate position on the underside of a seating surface 102 of a chair 100. The closer the blood vessels of a user 104 are to the body surface, the easier the measurement of pulse waves becomes. In this connection, blood vessels are running near the body surface at the back of the thighs in areas near the knees. Accordingly, the piezoelectric vibration sensor 110 is installed on the front-end side of the seating surface 102 so that pulse waves are transmitted to the piezoelectric vibration sensor 110 as shown by the arrow FP in the figure. On the other hand, ballistocardiac waves propagate primarily through the bones of the user 104, and are thus transmitted to the piezoelectric vibration sensor 110 as shown by the arrow FB.

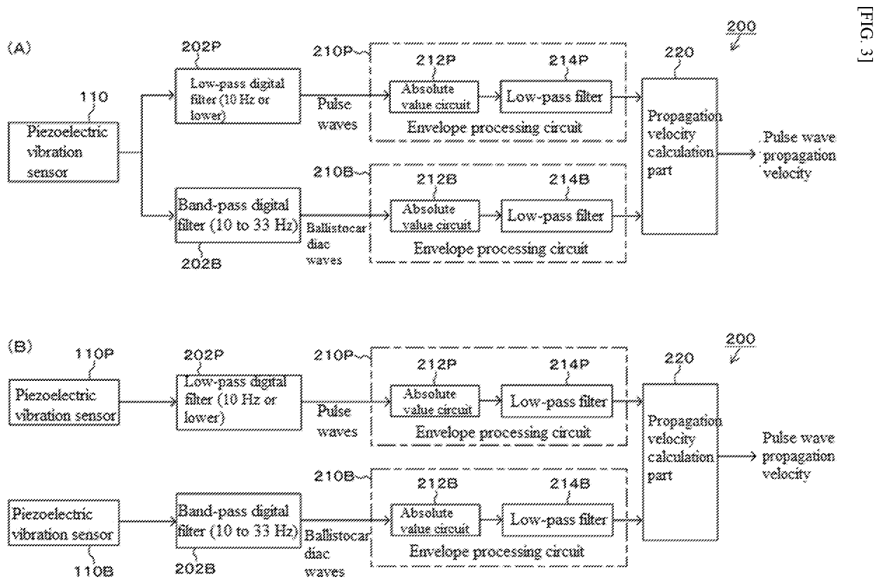

A measurement circuit 200 shown in FIG. 3 (A) is connected to the piezoelectric vibration sensor 110, while a low-pass digital filter 202P and a band-pass digital filter 202B are connected to the vibration waveform output side of the piezoelectric vibration sensor 110. The output sides of the two digital filters 202P, 202B are connected to envelope-processing circuits 210P, 210B, respectively. The envelope-processing circuit 210P is constituted by an absolute value circuit 212P and a low-pass filter 214P, while the envelope-processing circuit 210B is constituted by an absolute value circuit 212B and a low-pass filter 214B. The output sides of these envelope-processing circuits 210P, 210B are each connected to a velocity calculation part 220.

Among these, the low-pass digital filter 202P is a filter for isolating the pulse wave components, or components with a frequency range of 0 to 4 Hz (4 Hz or lower), from the output vibration waveforms of the piezoelectric vibration sensor 110. The band-pass digital filter 202B is a filter for isolating the ballistocardiac components, or components with a frequency range of 10 to 33 Hz, from the output vibration waveforms of the piezoelectric vibration sensor 110. The sampling frequency is set to 1000 Hz, for example.

The envelope-processing circuits 210P, 210B are circuits that take the absolute values of input signals using the absolute value circuits 212P, 212B, while also filtering them using the low-pass filters 214P, 214B, to obtain envelopes. The velocity calculation part 220 has a function to calculate the pulse wave propagation velocity PWV based on the outputs from the envelope-processing circuits 210P, 210B, to perform:

a. peak detection from envelopes of the output signal waveforms of the envelope-processing circuits 210P, 210B; and

b. calculation of pulse wave propagation velocity PWV based on the difference between the peak of an envelope of pulse waves GP and the peak of an envelope of ballistocardiac waves GB.

The calculation of pulse wave propagation velocity in b above is performed as follows, for example. While the velocity of ballistocardiac waves GB is sonic and approx. 1 km/s when propagating through bones, the velocity of pulse waves GP is approx. 10 m/s, which means that, when the two are compared, the velocity of ballistocardiac waves GB is close to infinity. As a result, the pulse wave GP is considered to have departed the heart at the time the ballistocardiac wave GB is detected by the piezoelectric vibration sensor 110, and therefore the pulse wave GP is considered to have propagated through its path in the human body within the time after the ballistocardiac wave GB was detected until the pulse wave GP is detected. Accordingly, pulse wave propagation velocity PWV is expressed, with respect to the blood vessel length from the heart to the back of the knee over the path through which the pulse wave GP travels, as follows: “Pulse wave propagation velocity (PWV)=Blood vessel length/(Time difference between the pulse wave and the ballistocardiac wave).” The time difference between the pulse wave and the ballistocardiac wave equals the time difference between the peak of the pulse wave GP and the ballistocardiac wave GB as obtained by envelope processing. It should be noted that the blood vessel length can be known using, for example, a method of measuring the body surface with a measuring tape based on an arterial blood vessel layout obtained beforehand by radiograph. Alternatively, it can be estimated from the person's height by obtaining the correlation between the blood vessel lengths of multiple people obtained as described above, and their heights.

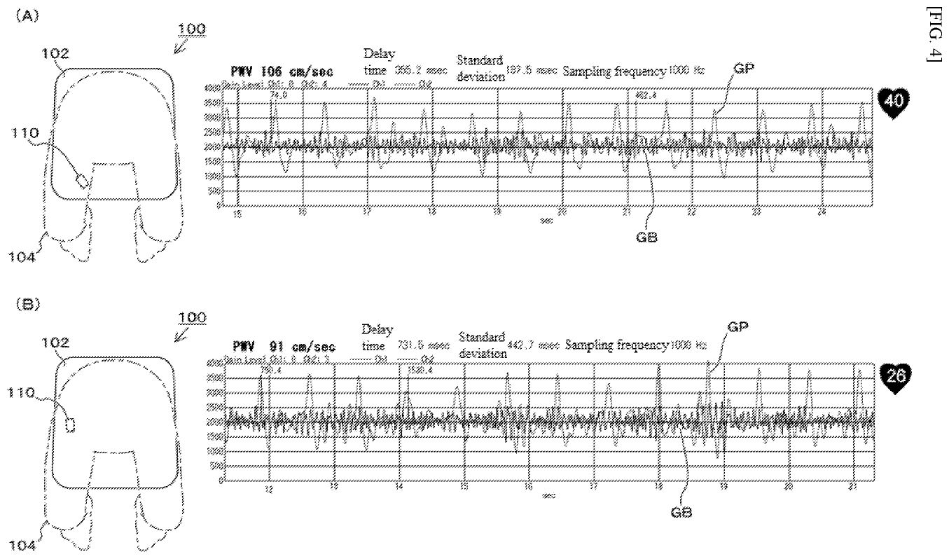

Next, the operations in this example are explained by also referring to FIGS. 4 to 6. The pulse waves and ballistocardiac waves that generate based on the beating of the heart of the user 104 propagate, through the seating surface 102 and the user 104, to the piezoelectric vibration sensor 110 attached to the seating surface 102 (refer to the arrows FP, FB in FIG. 2). At the piezoelectric vibration sensor 110, the transmitted vibrations are converted to electrical signals and output. These vibration waveforms are filtered by the low-pass digital filter 202P and the band-pass digital filter 202B, with the pulse wave range components output from the low-pass digital filter 202P and the ballistocardiac wave range components output from the band-pass digital filter 202B.

FIG. 4 shows examples of signal waveforms. FIG. 4 (A) shows a placement of the piezoelectric vibration sensor 110 at a relatively front-end side of the seating surface 102, while FIG. 4 (B) shows a placement of the piezoelectric vibration sensor 110 at a relatively rear-end side of the seating surface 102. GP indicates pulse waves, while GB indicates ballistocardiac waves. Looking at these graphs, clearly the pulse waves GP and ballistocardiac waves GB are both measured under either sensor placement, although there are some differences.

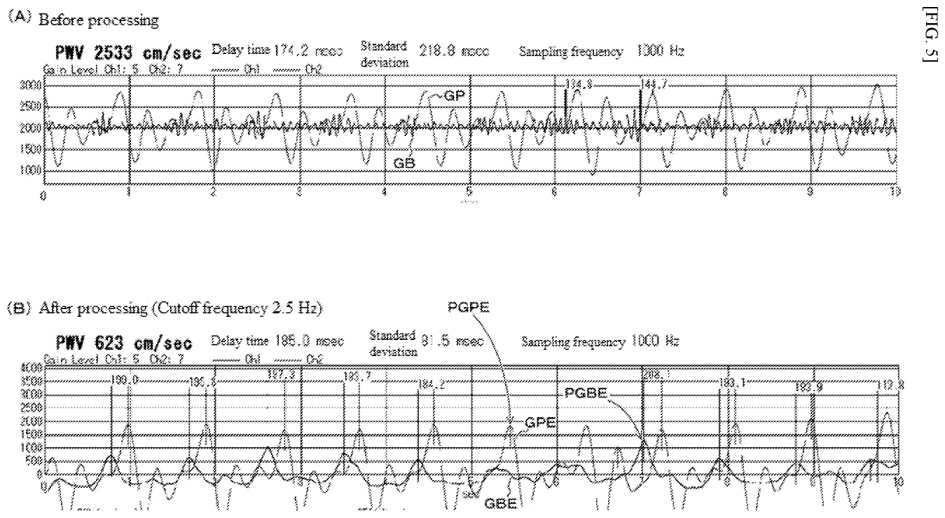

The pulse waves GP and ballistocardiac waves GB obtained by the piezoelectric vibration sensor 110 are input to the envelope-processing circuits 210P, 210B, respectively, for envelope detection. To be specific, the absolute values of input signals are obtained by the absolute value circuits 212P, 212B, and filtered by the low-pass filters 214P, 214B. For example, envelope-processing the pulse waves GP and the ballistocardiac waves GB shown in FIG. 5 (A) produces the envelopes GPE, GBE shown in FIG. 5 (B), respectively. The signals of these envelopes GPE, GBE are input to the velocity calculation part 220, where the difference between the peak PGPE of the envelope GPE of pulse waves GP and the peak PGBE of the envelope GBE of ballistocardiac waves GB is obtained, and additionally the pulse wave propagation velocity PWV is calculated based on this difference.

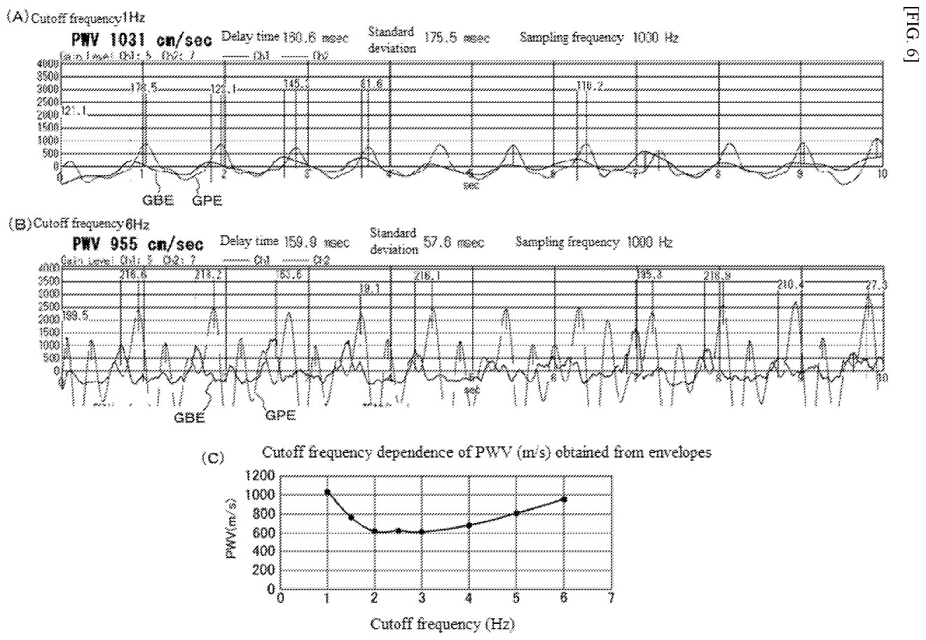

It should be noted that, in this case, how the cutoff frequencies are set for the low-pass filters 214P, 214B of the envelope-processing circuits 210P, 210B changes the value of pulse wave propagation velocity PWV. While FIG. 5 (B) described above assumes a cutoff frequency of 2.5 Hz, changing it to 1 Hz results in FIG. 6 (A), while changing it to 6 Hz results in FIG. 6 (B). This relationship between the cutoff frequency and the pulse wave propagation velocity PWV is shown in FIG. 6 (C). From the result of this graph, a cutoff frequency of 1.5 Hz or lower decreases the signal strengths of both the pulse wave GP and ballistocardiac wave GB signals, thereby preventing the correct value of peak time difference from being obtained. When the cutoff frequency exceeds 4 Hz, on the other hand, the envelope processing of ballistocardiac waves GB becomes insufficient, and this, in turn, prevents the correct pulse wave propagation velocity from being obtained. For this reason, preferably the cutoff frequency is set to 1.5 Hz or higher but no higher than 4 Hz.

Thus, according to this example, pulse waves and ballistocardiac waves are range-sampled from the output vibration waveforms of the piezoelectric vibration sensor 110 attached to the chair 100, and the peaks of their envelopes are utilized to calculate the pulse wave propagation velocity, which achieves the following:

a. pulse wave propagation velocity can be obtained in a favorable manner without having to attach the piezoelectric vibration sensor directly to the human body; and

b. pulse waves and ballistocardiac waves can be measured simultaneously using only one piezoelectric vibration sensor, and based on the measured results, pulse wave propagation velocity can be obtained in a favorable manner.

### Example 2

Next, Example 2 is explained by referring to FIG. 3 (B). In Example 1 described above, one piezoelectric vibration sensor 110 was used; in this example, however, piezoelectric vibration sensors 110P, 110B are connected to the low-pass digital filter 202P and the band-pass digital filter 202B, respectively, as shown in the figure. While this increases the number of piezoelectric vibration sensors compared to Example 1 above, the piezoelectric vibration sensors can each be installed in the vicinity of the underside of the tailbone highly sensitive to ballistocardiac waves, and in the vicinity of the back of the knee highly sensitive to pulse waves, which produces the benefit of further improvement in signal quality.

It should be noted that the present invention is not limited to the aforementioned examples and that various modifications may be added to the extent that the results do not deviate from the key points of the present invention. For example, the present invention also includes the following:

(1) In the aforementioned examples, the piezoelectric vibration sensor is attached to the seating surface of a chair, which is an item used universally by people in everyday life; however, it may also be attached to various things such as armrests, seatbacks, beds, pillows and other bedding articles, so long as the attached surface is contacted by people.

(2) The circuit configuration shown in FIG. 3 may also take various forms of performing the same signal processing, such as performing calculations using a computer program, for example.

### Industrial Field of Application

According to the present invention, pulse waves and ballistocardiac waves are isolated, by the filtering means, from the output vibration waveforms of the piezoelectric vibration sensor installed on a surface contacted by the human body, and these waves are utilized to calculate the pulse wave propagation velocity; accordingly, the pulse wave propagation velocity can be obtained in a favorable manner without having to attach the piezoelectric vibration sensor directly to the human body, which is suitable for the medical field.

### DESCRIPTION OF THE SYMBOLS

10, 12: Piezoelectric vibration sensor
100: Chair
102: Seating surface
104: User
110, 110P, 110B: Piezoelectric vibration sensor
200: Measurement circuit
202B: Band-pass digital filter
202P: Low-pass digital filter
210P, 210B: Envelope-processing circuit
212P, 212B: Absolute value circuit
214P, 214B: Low-pass filter
220: Velocity calculation part
GB: Ballistocardiac wave
GP: Pulse wave
GPE, GBE: Envelope
PGPE, PGBE: Peak
PWV: Pulse wave propagation velocity

1. A pulse wave propagation velocity measurement device that obtains a human pulse wave propagation velocity based on output vibration waveforms of a piezoelectric vibration sensor attached to a surface adapted to be contacted by a human body, the pulse wave propagation velocity measurement device characterized by comprising:
a first filtering means for isolating pulse waves from the output vibration waveforms of the piezoelectric vibration sensor;
a second filtering means for isolating ballistocardiac waves from the output vibration waveforms of the piezoelectric vibration sensor; and
a calculation means for calculating pulse wave propagation velocity by utilizing the pulse waves obtained by the first filtering means and the ballistocardiac waves obtained by the second filtering means.

2. The pulse wave propagation velocity measurement device according to claim 1, characterized in that the first filtering means isolates frequency range components of 4 Hz or lower from the output vibration waveforms of the piezoelectric vibration sensor, and the second filtering means isolates frequency range components of 10 Hz or higher but no higher than 33 Hz from the output vibration waveforms of the piezoelectric vibration sensor.

3. The pulse wave propagation velocity measurement device according to claim 1, characterized in that the calculation means comprises:
a first envelope-processing means for obtaining envelopes of the pulse waves obtained by the first filtering means, and a second envelope-processing means for obtaining envelopes of the ballistocardiac waves obtained by the second filtering means; and
a velocity calculation means for calculating the pulse wave propagation velocity by utilizing peaks of the envelopes obtained by the first and second envelope-processing means.

4. The pulse wave propagation velocity measurement device according to claim 3, characterized in that the velocity calculation means obtains a time difference between the pulse wave and the ballistocardiac wave from the peaks of the envelopes, and also calculates “Pulse wave propagation velocity=Blood vessel length/(Time difference between the pulse wave and the ballistocardiac wave)” with respect to a blood vessel length of a path along which the pulse waves travel.

5. The pulse wave propagation velocity measurement device according to claim 3, characterized in that the first and second envelope-processing means are each constituted by an absolute value circuit and a low-pass filter, and a cutoff frequency of the low-pass filter is set to 1.5 Hz or higher but no higher than 4 Hz.

6. The pulse wave propagation velocity measurement device according to claim 1, characterized in that it obtains the pulse waves and ballistocardiac waves from the output vibration waveforms of one piezoelectric vibration sensor.

7. The pulse wave propagation velocity measurement device according to claim 1, characterized in that it obtains the pulse waves and ballistocardiac waves from the output vibration waveforms of different piezoelectric vibration sensors.

8. A method for pulse wave propagation velocity measurement that obtains a human pulse wave propagation velocity based on output vibration waveforms of a piezoelectric vibration sensor attached to a surface adapted to be contacted by a human body, the method for pulse wave propagation velocity measurement characterized by comprising:
a first step in which pulse waves are isolated from the output vibration waveforms of the piezoelectric vibration sensor;
a second step in which ballistocardiac waves are isolated from the output vibration waveforms of the piezoelectric vibration sensor; and
a third step in which the pulse wave propagation velocity is calculated by utilizing the pulse waves obtained in the first step and the ballistocardiac waves obtained in the second step.

9. The method for pulse wave propagation velocity measurement according to claim 8, characterized in that the first step isolates frequency range components of 4 Hz or lower from the output vibration waveforms of the piezoelectric vibration sensor, and the second step isolates frequency range components of 10 Hz or higher but no higher than 33 Hz from the output vibration waveforms of the piezoelectric vibration sensor.

10. The method for pulse wave propagation velocity measurement according to claim 8, characterized in that the third step comprises:
a first envelope-processing step in which envelopes of the pulse waves obtained in the first step are obtained, and a second envelope-processing step in which envelopes of the ballistocardiac waves obtained in the second step are obtained; and
a velocity calculation step in which the pulse wave propagation velocity is calculated by utilizing peaks of the envelopes obtained in the first and second envelope-processing steps.

11. The method for pulse wave propagation velocity measurement according to claim 10, characterized in that the velocity calculation step obtains a time difference between the pulse wave and the ballistocardiac wave from the peaks of the envelopes, and also calculates “Pulse wave propagation velocity=Blood vessel length/(Time difference between the pulse wave and the ballistocardiac wave)” with respect to a blood vessel length of a path along which the pulse waves travel.

12. The method for pulse wave propagation velocity measurement according to claim 8, characterized in that it obtains the pulse waves and ballistocardiac waves from the output vibration waveforms of one piezoelectric vibration sensor.

13. The method for pulse wave propagation velocity measurement according to claim 8, characterized in that it obtains the pulse waves and ballistocardiac waves from the output vibration waveforms of different piezoelectric vibration sensors.

14. The pulse wave propagation velocity measurement device according to claim 2, characterized in that the calculation means comprises:
a first envelope-processing means for obtaining envelopes of the pulse waves obtained by the first filtering means, and a second envelope-processing means for obtaining envelopes of the ballistocardiac waves obtained by the second filtering means; and
a velocity calculation means for calculating the pulse wave propagation velocity by utilizing peaks of the envelopes obtained by the first and second envelope-processing means.

15. The pulse wave propagation velocity measurement device according to claim 2, characterized in that it obtains the pulse waves and ballistocardiac waves from the output vibration waveforms of one piezoelectric vibration sensor.

16. The pulse wave propagation velocity measurement device according to claim 2, characterized in that it obtains the pulse waves and ballistocardiac waves from the output vibration waveforms of different piezoelectric vibration sensors.

17. The pulse wave propagation velocity measurement device according to claim 3, characterized in that it obtains the pulse waves and ballistocardiac waves from the output vibration waveforms of one piezoelectric vibration sensor.

18. The method for pulse wave velocity measurement according to claim 9, characterized in that the third step comprises:
a first envelope-processing step in which envelopes of the pulse waves obtained in the first step are obtained, and a second envelope-processing step in which envelopes of the ballistocardiac waves obtained in the second step are obtained; and
a velocity calculation step in which the pulse wave velocity is calculated by utilizing peaks of the envelopes obtained in the first and second envelope-processing steps.

19. The method for pulse wave velocity measurement according to claim 9, characterized in that it obtains the pulse waves and ballistocardiac waves from the output vibration waveforms of one piezoelectric vibration sensor.

20. The method for pulse wave velocity measurement according to claim 9, characterized in that it obtains the pulse waves and ballistocardiac waves from the output vibration waveforms of different piezoelectric vibration sensors.
